(19) United States
(12) Patent Application Publication
(10) Pub. No.: US 2019/0290482 A1
(43) Pub. Date: Sep. 26, 2019

(54) DENSE PHASE MATERIAL TRANSPORT IN PULMONARY SYSTEM

(71) Applicant: Qool Therapeutics, Inc., Menlo Park, CA (US)

(72) Inventors: Jeff G. Haydon, Menlo Park, CA (US); Edward J. Hayes, III, San Jose, CA (US)

(73) Assignee: Qool Therapeutics, Inc., Menlo Park, CA (US)

(21) Appl. No.: 16/436,169

(22) Filed: Jun. 10, 2019

Related U.S. Application Data
(63) Continuation of application No. PCT/US17/65628, filed on Dec. 11, 2017.
(60) Provisional application No. 62/433,642, filed on Dec. 13, 2016.

(51) Int. Cl. A61F 7/12; A61M 16/10; A61M 16/14; A61M 39/22

(52) U.S. Cl. CPC A61F 7/12; A61M 16/109; A61M 2205/3606; A61M 39/22; A61M 2202/066; A61M 16/14

(57) ABSTRACT

Systems for dense phase transport of frozen and other particles to the respiratory system include a particle source, a delivery chamber for metering boluses of the particles from the source, and a transfer tube for fluidized transport of the particles to a patient interface. A controller may be provided to adjust the rate and amount of the bolus deliver to a patient to control core body temperature and for other purposes.

### BACKGROUND OF THE INVENTION

This application is a continuation of PCT Application No. PCT/US17/65628 (Attorney Docket No. 32138-713601), filed Dec. 11, 2017, which claims the benefit of Provisional Patent Application 62/433,642 (Attorney Docket No. 32138-713.101), filed on Dec. 13, 2016, the full disclosure of which is incorporated herein by reference.

### BACKGROUND OF THE INVENTION

The present invention relates generally to medical systems and methods. More particularly, the present invention relates to systems and methods for inducing hypothermia in patients and optionally delivering medications to the patient as hypothermia is being induced.

Methods and systems for effecting systemic hypothermia and optionally administering drugs by delivering ice and other frozen particles to the lungs, abdominal cavity, and other target sites of a patient have been described and implemented by Qool Therapeutics, Inc., assignee of the present application. For example, WO/2016/138045 teaches that frozen saline particles (FSP) and other frozen particles can be delivered as an aerosol or as a series of boluses to a patient through a patient interface, such as an endotracheal tube. Other patents and patent applications assigned to Qool Therapeutics, Inc. further teach that drugs, biologicals, and other active agents, can be delivered to the lungs concurrently with FSP delivery. See, for example, US20120167878; U.S. Pat. No. 8,100,123; and WO/2017/132609.

While the systems and methods described in these patents and published applications are effective, there remains a need for alternative FSP delivery and hypothermia systems which provide alternative modes of FSP delivery. In particular, the availability of different FSP delivery systems and delivery protocols can improve the ability to control density of the FSP and other frozen particles streams, slurries, and boluses is desirable. Density control can, in turn, help control the dispersability and penetrability of the FSP as they enter the lungs and other target sites.

### SUMMARY OF THE INVENTION

The following invention describes apparatus and methods for delivering large amounts of one or more solid phase source materials, such as frozen saline particles (FSP), to the respiratory system. The apparatus makes use of interactions between the constituting particles to enable efficient and effective delivery of powder or powder-like materials to the respiratory system. Conventional technologies making use of aerosol physics, such as nebulizers, are generally limited to a maximum material delivery rate of approximately 0.1 grams per breath cycle to the lungs. Furthermore, only a small fraction—generally less than 20%—of the material is successfully delivered to the targeted region of the lungs. By designing the delivery mechanism to obtain a desired powder and flow characteristics, metered amounts of material, typically ranging from 0.1 gram to 3 grams or more per inhalation cycle may be safely and efficiently transported into the respiratory system. In certain instances, a single or multiple larger boluses, up to 100 grams, may be transported into the respiratory system. Modulating machine settings/hardware configuration and/or powder properties such as bulk density, particle size distribution, or morphology allows target regions for source material(s) deposition to be varied enabling region specific therapies for areas such as the trachea, bronchial tree, upper airways, lower airways, and alveoli, or some combination thereof The source material(s) delivered to the respiratory system may include particulate/powdered solids, such as frozen aqueous solutions, frozen saline, frozen lactated Ringer's solution, water with or without the addition of additional elements (for example, salts), drugs, and biologics (for example, whole and/or parts of and or derivatives of stem cells, which may be frozen), among others. A single source material or multiple source materials may be delivered singularly, combined, sequentially, or in other combinations, to the respiratory system and/or over a range of transfer regimes, including dilute phase (aerosol) through dense phase—plug flow.

The currently accepted and most common method of drug/particle delivery to the lungs is via aerosolization. Aerosolization is the process of converting some physical substance (liquid or powder in nature) into particles that are light enough to be carried by gaseous fluids or air. The most common approach to aerosolizing a liquid substance is nebulization. An aerosol—or a collection of particles suspended in a gaseous fluid—is limited in the amount of material (fluid or powder in nature) it can carry and transport. In the bulk powder industry, powder aerosols are sometimes called “lean phase” or “dilute phase” suspensions; as these names imply, aerosols cannot carry a large number of particles or suspend a substantial amount of mass. If there are more particles in a gaseous mixture than can be supported in an aerosolized nature, the increased rate of particle interaction may cause agglomeration and cause the powder (or fluid) to fall out of suspension. The maximum mass of particles (fluid or powder) that can be suspended in an aerosol is determined by a number of factors including, but not limited to: particle size, particle density, carrier gas density, carrier gas velocity, and particle velocity. As a consequence of the dilute nature of aerosols, it takes an exceptionally large volume of gas to transport a relatively small quantity of material.

One aspect of the present invention makes it possible to utilize inter-particle interactions to overcome conventional delivery limitations on individual particle mass, size, and bulk mass delivery (grams delivered per breath). Tuning an aerosol for delivery into the pulmonary system typically involves reducing the momentum of particles allowing the suspended aerosol to follow the flow of air through the bronchi. If particles are outside optimal size ranges, they can exhibit challenging bulk properties resulting in decreased ability to handle, meter, or aerosolize. Particles that are too small may not successfully deposit in the pulmonary system (expelled from patient during exhalation), or may exhibit increased rates of particle interaction resulting in agglomeration and limited delivery rates. If particles are too massive, momentum is likely to prevent them from being conveyed by carrying gasses resulting in undesired deposition in the upper airways, limiting therapeutic benefit. Aerosol particles for deposition in the pulmonary system are commonly less than 10 μm in diameter. The present invention can efficiently and reliably deliver a wide range of particle sizes at significantly higher maximum rates of delivery.

Commonly, aerosols are only able to deliver up to approximately 0.1 grams of material per actuation or inspiration cycle, well below the levels that can be achieved with the present invention. Schematic representations of (A) dilute phase (aerosol), (B) dilute phase—strand flow, (C) dense phase—dune flow, and (D) dense phase—plug flow particle transport are shown in FIGS. 1A-1D, respectively.

Aerosols typically suffer from low bioavailability due to deposition in untargeted regions—resulting in inefficient or unsuccessful therapy. Less than 20% bioavailability is common for inhaled drugs. By utilizing the present invention, it is possible to significantly increase bioavailability of materials delivered to the respiratory system.

Delivery systems according to the present invention are capable of delivering one or more source materials to the respiratory system in a higher density phase compared to dilute phase transport (aerosol). This increased density of material(s) is transported using a smaller amount of carrier gas, traveling at a lower velocity, to transport larger quantities of material(s). Rather than a constant stream of material(s), the material(s) is moved in a more discrete unit—essentially dense waves or plugs of material(s).

The delivery system of the present invention is capable of delivering a metered amount of material(s) to the respiratory system using dense phase transport. Unlike in aerosols, particles in dense phase transport are not predominantly suspended by gas. Inter-particle interactions are responsible for much of the bolus support in dense phase transport. Because of this, conventional restrictions on particle size, mass, density, and shape may be reduced or eliminated when delivering to the respiratory system. Dense phase—plug flow transport has the ability to transport and deliver more than 10 times the material(s) than conventional aerosol delivery methods for the same quantity of carrier gas.

The delivery system of the present invention is capable of delivering a metered amount of material(s) in dilute phase—strand flow and dense phase—dune flow transport.

The delivery system of the present invention using dilute phase—strand flow and/or dense phase transport in addition to enabling larger per breath delivery rates when compared to aerosolized particle delivery, also provides for varying the delivery mechanics and dynamics. When properly implemented, these mechanics provide new delivery options for targeted drug and whole and/or parts of and or derivatives of stem cells, allow morphology changes to the source material(s) particles (or may relax particles size constraints), and when desired, increase delivery efficiency to distal regions of the lungs by reducing the particle-bronchial tree interactions, etc—providing improved delivery dynamics to the respiratory system.

In further aspects of the present invention, there are provided methods for delivering material(s) to the respiratory system in a higher density phase compared to dilute phase (aerosol) transport. Such methods generally comprise the steps of (A) metering or selecting a desired bolus of a material(s) to be delivered, and (B) delivering the material(s) to the respiratory system in a higher density phase compared to dilute phase (aerosol) transport. Further aspects, details, examples and embodiments of the invention will be appreciated by those of skill in the art upon reading the detailed description of the invention set forth below.

In a first specific aspect, the present invention provides a method for producing a series of frozen solid particle (FSP) boluses for delivery to a patient interface, such as an endoscope, breathing mask, or other interface intended to deliver breathing and carrier gases to a patient's lungs. The method comprises providing a source of FSP, transferring a metered bolus of FSP from the source into a delivery chamber, and fluidizing the metered bolus of FSP. The fluidized, metered bolus of FSP is transferred to the patient interface, typically through a transfer tube as described below, and the steps of transferring the metered bolus to the delivery chamber, fluidizing the metered bolus, and transferring the fluidized metered bolus to the patient interface are typically repeated in order to provide a series of discrete FSP boluses into the patient interface.

In the specific embodiments of this method, providing the source of FSP may comprise storing a volume of preformed FSP in a “hopper” or other receptacle. Optionally, the hopper may have cooling, mixing, ventilating, and other environmental controls to maintain the FSP is a flowable, fluidizable (dispersible) condition prior to transfer to the delivery chamber. Alternatively, the providing step may comprise comminuting, typically grinding, dicing, crushing, or otherwise forming a mass of FSP particles from an aggregate source of frozen or other FSP material. For example, a block or other large volume of frozen saline may be chipped, ground, chopped, or otherwise formed into particles in the desired size ranges described hereinbelow. As a third alternative, the source of FSP may comprise freezing a liquid source material into FSP in situ, e.g. by spraying the liquid source material into a chilled gas stream.

In other specific embodiments of this method, the bolus of FSP may be metered into a delivery chamber by flowing the FSP from the source into the delivery chamber and metering. Metering may be performed using a valve, such as an on-off valve, a rotary bolt valve, or the like, and in some instances the valve can be incorporated into the delivery chamber and/or act as a transfer interface between the FSP source and the delivery chamber. Typically, such metering will be effected by flowing the FSP from the source into the delivery chamber by gravity flow, pressurization of the source, or a combination of both. For example, the pressure within the FSP source may be raised, pressure within the delivery chamber may be reduced, or a combination of thereof may be used to create a differential pressurization to initiate or enhance flow from the FSP source into the delivery chamber. The delivery chamber may optionally have mixing and environmental controls similar to those described above for the storage hopper.

In still further specific embodiments of this method, the metered bolus of FSP from the delivery chamber is transferred to the patient interface through a transfer tube, typically a tube or tube-like component, more typically a tapered tube which is tapered to decrease in cross-sectional area in the direction from the delivery chamber toward the patient interface. For example, the cross-sectional area may decrease by 10% to 95% from an inlet end to an outlet end, preferably from 60% to 90%. Such tapering can compact the FSP as they flow from the delivery chamber to the patient interface to provide a desired densification. In such embodiments, fluidizing the metered bolus of FSP will typically comprise fluidizing within at least one of the delivery chamber and the transfer tube, typically fluidizing in both of the delivery chamber and the transfer tube. In some instances, the delivery chamber and the transfer tube may be integrated into a single unit. In some instances, the patient interface may be tapered to narrow in the direction away from the transfer tube.

In still additional specific embodiments, the methods further comprise controlling the conditions of fluidizing the metered bolus of FSP and of transferring the fluidized metered bolus of FSP to the patient interface in order to control a density of the FSP delivered to the interface. For example, the conditions may be controlled to cause a “strand” flow to the patient interface, as described further below. Alternatively, the conditions may be controlled to cause a “dune” flow to the patient interface, as described further below. Still further alternatively, the conditions may be controlled to cause a plug flow to the patient interface, as commonly understood and described further below.

In a second particular aspect, the present invention provides a method for lowering a core body temperature of a patient. The method comprises providing a source of FSP, transferring a metered bolus of FSP from the source into a delivery chamber, fluidizing the metered bolus of FSP, delivering the fluidized metered bolus of FSP to the patient's lungs, and optionally delivering a breathing gas to the patient's lungs. In some instances, the FSP can be introduced into the patient's lungs without separately ventilating the patient, typically when small volumes of FSP are being delivered, for example during drug delivery without systemic hypothermia. When inducing hypothermia, in contrast, a larger volume of the FSP will typically be dispersed in a breathing gas delivered to the patient's lungs such that the dispersed FSP melt in the lungs to lower a core body temperature of the patient. Thus, these methods usually further comprise delivering a breathing gas to the patient's lungs wherein the FSP are dispersed into the gas. More usually, the FSP are dispersed into the breathing gas in synchrony with the patient's breathing cycle, typically during inhalation but not during exhalation. In still other instances, the FSP may be delivered sequentially with either inhalation or exhalation during an induced (ventilated) or natural breathing cycle breathing.

In a specific embodiment of this method, the steps of the method are repeated in order to deliver a series of discrete FSP boluses into the patient's lungs to control the core temperature to a target temperature. Optionally, the fluidized FSP and the breathing gas may be delivered through separate conduits in a patient interface, where the FSP are dispersed in the breathing gas within the patient's lungs after release from the patient interface. Alternatively, the fluidized FSP and the breathing gas may be delivered at least partially through a common conduit in the patient interface, wherein the FSP disperse in the breathing gas prior to release from the patient interface. Further optionally, the breathing gas may contain a pharmaceutically or biologically active substance to be delivered to the patient together with the FSP.

In particular protocols of this method, the breathing gas may be delivered during at least a portion of some of the patient's inhalation cycles but typically not during the patient's exhalation cycles. Providing the source of FSP may comprise storing a volume of the preformed FSP in a hopper, comminuting an aggregate source of the FSP, and/or freezing a liquid source material into FSP in situ, all as generally described above in connection with the methods for producing metered FSP boluses.

In a third particular aspect, the present invention provides a system for lowering a core body temperature of a patient. The system comprises a source of FSP, a delivery chamber configured to receive metered boluses of FSP from the FSP source, a transfer tube having an inlet end and an outlet end, and a fluidizer configured to receive the metered bolus of FSP from the delivery chamber and to deliver the fluidized metered bolus of FSP to an inlet end of the transfer tube. Typically, the outlet end of the transfer tube is configured to detachably couple to a patient interface, where the patient interface is configured to deliver the fluidized, metered boluses of FSP into the patient's lungs to effect core body cooling. In other instances, the patient interface may be integrated with the transfer tube.

Specific embodiments of the systems of the present invention may further comprise a controller configured to adjust the amount and/or rate of FSP delivery from the FSP source to the delivery chamber and/or from the delivery chamber through the patient interface and thus to the patient. In this way, a target core temperature of the patient can be achieved and maintained by adjusting the amount and/or rate of FSP delivery using the controller. Conventional feedback and other known control algorithms may be used. Specific control protocols and algorithms are described in WO/2016/138045, the full disclosure of which is incorporated herein by reference.

Optionally, the systems of the present invention may further comprise a patient ventilator or other breathable gas source configured to deliver at least a portion of the gas output to the fluidizer(s). Alternatively or additionally, the ventilator may be further configured to deliver at least a portion of the breathable gas output to the delivery chamber. Still further additionally or alternatively, the ventilator may be configured to deliver at least a portion of the breathable gas output to the patient interface.

In further exemplary systems according to the present invention, the system will further include the patient interface. In such instances, the patient interface may comprise a single lumen configured to receive both the breathing gas and the FSP, typically or the FSP are fluidized and suspended within the breathing gas. Additionally or alternatively, the breathing gas may be delivered to the breathing tube through a line fluidizer. Alternatively or additionally, the patient interface may comprise a first lumen configured to receive a breathing gas and a second lumen configured to receive the FSP.

The controller of the systems of the present invention may be configured to implement any of the specific FSP protocols described above in connection with the methods of the present invention.

### BRIEF DESCRIPTION OF THE DRAWINGS

Various embodiments of the present invention will now be discussed with reference to the appended drawings. It should be appreciated that the drawings depict only typical embodiments of the invention and are therefore not to be considered limiting in scope.

FIG. 1A illustrates dilute phase (aerosol) particle transport.

FIG. 1B illustrates dilute phase—strand flow particle transport.

FIG. 1C illustrates dense phase—dune flow particle transport.

FIG. 1D illustrates dense phase—plug flow particle transport.

FIG. 2 is a schematic diagram of a frozen aqueous particle generator.

FIG. 3 is a first embodiment of a frozen aqueous particle delivery mechanism.

FIG. 4 is an alternate embodiment of a frozen aqueous particle delivery mechanism constructed in accordance with the principles of the present invention.

FIG. 5 is an alternate embodiment of a frozen aqueous particle delivery mechanism constructed in accordance with the principles of the present invention.

### DETAILED DESCRIPTION OF THE INVENTION

The following detailed description of the present invention focuses primarily on dense phase transport of frozen aqueous particles (FSP) 18, such as frozen saline particles or other source materials to the respiratory system. The systems described herein are capable of dense phase delivery of frozen aqueous particles 18 into the respiratory system. The systems described herein are further capable of delivering one or more source materials singularly, combined, sequentially, or in other combinations and at the same or different time points, to the respiratory system and/or over a range of transfer regimes, including dilute phase (aerosol) through dense phase—plug flow. The detailed description focuses on a generation and delivery system including such components as controls, patient interface 200, and ventilator 300, as required to enable dilute phase—strand flow and dense phase transport of frozen aqueous particles 18 to the lungs. The system of the present invention is not limited to dilute phase—strand flow and dense phase transport of frozen aqueous particles 18 to the lungs. The system of the present invention need not generate the source material or source materials, and may comprise only the delivery mechanism 100 and patient interface 200, and optionally the controller 195. For example, a source material, such as intact stem cells or portions thereof, which be frozen, may be introduced into the delivery mechanism 100 for delivery to the respiratory system using the present invention. Such intact or partial stem cells, which may be frozen, may be mixed with other source material(s), such as frozen aqueous particles 18, and delivered at the same time point, and/or they may not be mixed and delivered sequentially or at different time points.

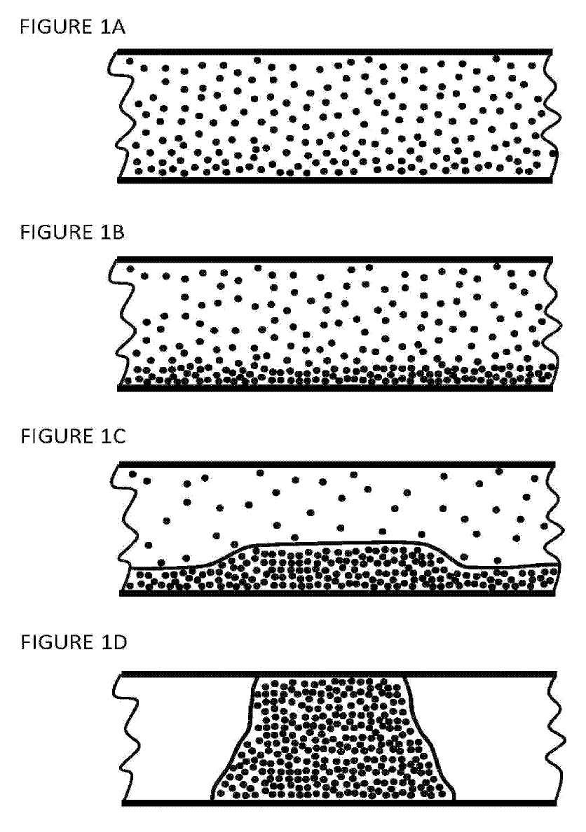

Various particle transport flows are depicted in FIGS. 1A-1D, for a sample particle. FIG. 1A depicts dilute phase (aerosol), which has comparatively high gas and particle velocities, with a relatively lower particle transport per volume of carrier gas. Dilute phase flow occurs when the majority of the materials are transported in suspension with the carrier gas. As such, for the majority of the material to be in suspension, there needs to be a lot of carrier gas and relatively few materials. To maintain a dilute phase transport regime, the carrier gas velocity must be kept relatively high.

FIG. 1B depicts dilute phase—strand flow. Dilute phase—strand flow occurs when, due to material properties or velocity of the carrier gas, causes some of the materials to be transported as a ‘sliding strand’ along the bottom of the transfer tube or other patient interface, even in dilute phase.

FIG. 1C depicts dense phase—dune flow. Dense phase—dune flow is where materials drop out of suspension, but typically remain fluidized by the carrier gas, and are transported as a fluidized dune.

FIG. 1D depicts dense phase—plug flow which has relatively lower carrier gas and material velocities and greater material transport per volume of carrier gas than dilute phase (aerosol). Dense phase—plug flow is typically where the material being transported has sufficient carrier gas permeability to transport as a plug of material—one that generally fills a cross-section of the transfer tube—and typically is delivered in discrete “packets” of material. Dense phase—plug flow may be intermittent and by design, stop and restart transport of a “packet” within the transfer tube.

Bulk density, as used herein, refers to the mass of a group of particles divided by the volume that the particles and the gas between that group of particles occupy. For example, if an increase in fluidization gas or carrier gas causes a given mass of particles to occupy a greater volume, the bulk density of that group of particles is decreased.

Particle size distributions, cohesive properties, morphology (spherical shape versus non-spherical shape), and transport phase may be engineered to enable particle deposition in selectively or in a combination of the trachea, bronchial tree, upper airways, lower airways, and alveoli, enabling multiple regions for targeted therapy.

Frozen aqueous particles 18 are generated by freezing liquid aqueous solutions 12, such as normal (0.9%) saline, and as required, modifying that frozen aqueous solution 14 to size and shape to achieve the desired overall properties. Freezing liquid aqueous solution 12 may be accomplished by reducing the temperature below its freezing point using, for example, cryogenic materials (e.g. liquid nitrogen), thermoelectric cooling, mechanical refrigeration, vacuum, or a combination thereof. The rate of cooling and the temperature at which the liquid aqueous solution 12 is frozen can affect the properties of the frozen aqueous solution 14. Larger quantities of liquid aqueous solution 12 that are frozen slowly can have very different properties than very small amounts of liquid aqueous solution 12 frozen very rapidly. Any changes in properties caused by a different cooling rate may be accounted for in the system by adjusting, for example, the operating temperature or the mechanisms by which each subsystem or the overall system operates.

Once the liquid aqueous solution 12 is frozen, it may be desirable to then modify the shape and size of the frozen aqueous solution 14 to obtain the desired properties of frozen aqueous particles 18. The frozen aqueous solution 14 may be modified, such as by grinding, milling, pulverizing, chopping, etc. to form the desired frozen aqueous particles 18 for delivery to the respiratory system. The size and shape of the frozen aqueous particles 18 in combination with system operating parameters, such as fluidization levels, pressures, temperatures, as well system physical parameters, such as dimensions, materials, taper(s) 180, can all be modified to optimize targeted delivery, safety, and efficacy.

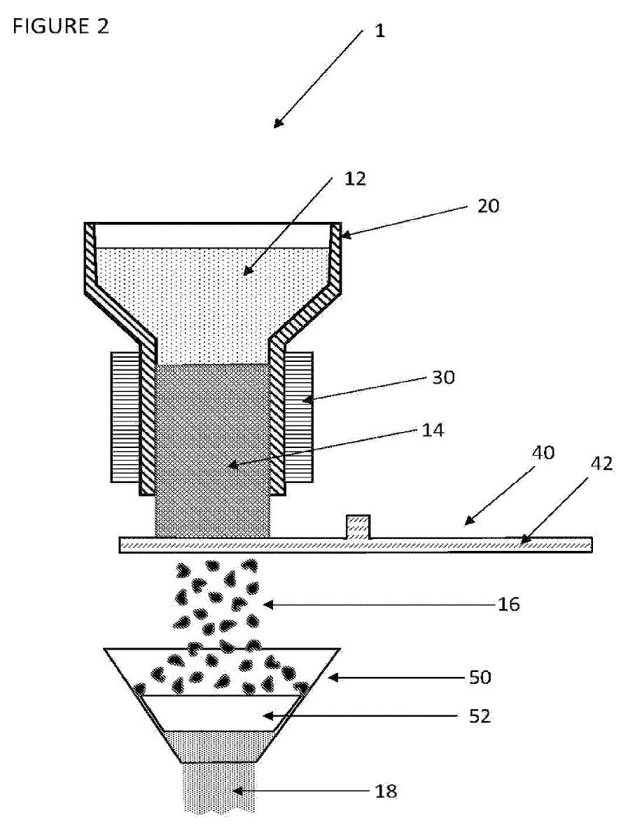

FIG. 2 is a schematic diagram of a frozen aqueous particle generator 1 apparatus for freezing a liquid aqueous solution 12, such as liquid saline, and grinding and milling the frozen aqueous solution 14 to the desired shape and size resulting in frozen aqueous particles 18. Liquid aqueous solution 12 from a suitable source, such as a container, IV bag, etc. (not shown) is introduced into the reservoir 20. The liquid aqueous solution 12 flows into the region of the thermoelectric/electromechanical cooler 30 and the temperature of the liquid aqueous solution 12 is lowered sufficiently to freeze the liquid aqueous solution 12 into frozen aqueous solution 14. The volume of liquid aqueous solution 12 and the temperature differential may be controlled to provide varying properties of the frozen aqueous solution 14. Optionally, the frozen aqueous solution 14 is ground by the course grinder 40 into course ground frozen aqueous particles 16 exhibiting an intermediate size and shape.

The course ground frozen aqueous particles 16 properties may be adjusted by changing the grinding parameters (speed, feed rate, etc), temperature, configuration of the course grinder blades 42, etc. The course ground frozen aqueous particles 16 can optionally be fed into a mill 50, which mills the course ground frozen aqueous particles 16 into frozen aqueous particles 18, which exhibit the final size and shape as desired for delivery into the respiratory system. The final properties of the frozen aqueous particles 18 may be modified by adjusting the milling parameters, such as the speed, feed rate, temperature, configuration of the mill cutter 52, etc. Other techniques to obtain the final properties of the frozen aqueous particles 18 may be used. As desired, the frozen aqueous particles 18 may be made directly from the liquid aqueous solution 12 by directly freezing into the desired size and shape, such as by cryogenic freezing of small units of liquid aqueous solution 12. The frozen aqueous solution 14 may also be converted to the final desired size and shape of frozen aqueous particles 18 or through one or more intermediary steps. The nominal size and shape of the frozen aqueous particles 18 is generally between 0.1 μm and 1000 μm, more suitably within a range of 0.1 μm to 500 μm, and more preferably 0.1 μm to 300 μm

The frozen aqueous particles 18 may be produced at varying rates and/or timepoints. Examples include; the amount of frozen aqueous particles 18 being generated is close the amount of frozen aqueous particles 18 being delivered, which may be varied based on delivery rate; a quantity of frozen aqueous particles 18 are generated and stored or maintained in the hopper reservoir 120 or other system component or storage device at any point in time; a quantity of frozen aqueous particles 18 are generated and maintained in the hopper reservoir 120 or other system component or storage device while the rate of new frozen aqueous particles 18 generation is closely tied to the amount of frozen aqueous particles 18 being delivered. Additionally, frozen aqueous particles 18 may be generated and stored for future use and when desired, introduced into the hopper 110, hopper reservoir 120, or other system component. Liquid aqueous solution 12 may also be frozen at any point in time as, for example, cubes, ingots, or some other size larger or equal to the coarse grinding requirements, then coarse ground to final size, to be stored or used at that time.

At any point in time after or during production of the frozen aqueous particles 18, the frozen aqueous particles 18 may be sorted, for example, based on size and/or properties, by—sieving, vortex, or other classification method. This enables the ability to include or exclude certain frozen aqueous particles 18.

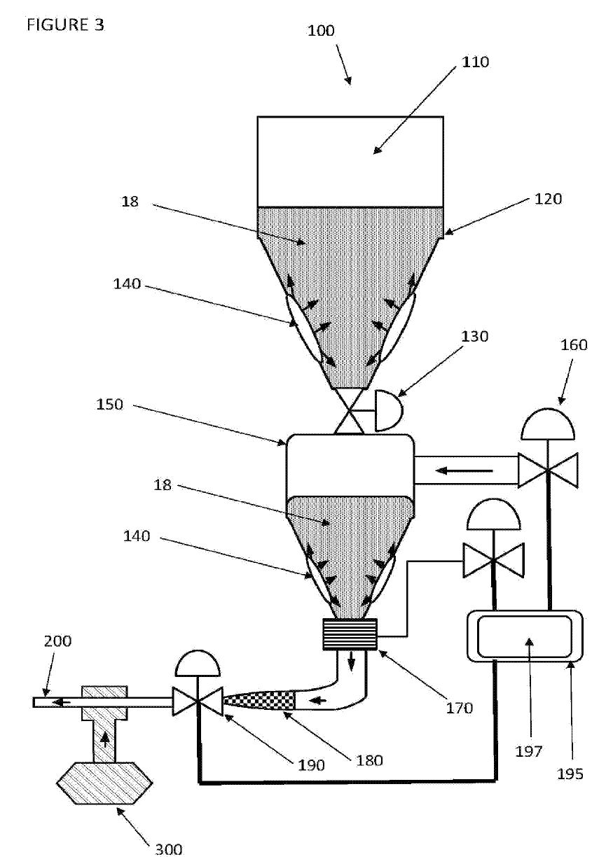

Upon producing the desired frozen aqueous particles 18, they may be stored for future use or transported to or fed directly to the delivery mechanism 100. A delivery mechanism 100 for delivering particulate material(s), such as frozen aqueous particles 18, to the respiratory system using dilute phase—strand flow and dense phase transport is depicted in FIG. 3. A hopper 110 is desired to receive and store the frozen aqueous particles 18 from the frozen aqueous particle generator 1 and hold them for metering and delivery. The frozen aqueous particles 18 are metered into the hopper 110 and reside in the hopper reservoir 120. The frozen aqueous particles 18 in the hopper reservoir 120 may be fluidized by injecting a gas, such as air, which may be cooled, through one or more fluidizing diffusers 140, ports, nozzles, etc., into the frozen aqueous particles 18. Fluidization may be used to enable the frozen aqueous particles 18 to flow in a manner similar to that of a liquid or to otherwise reduce cohesion and improve or increase flow. This improves the ability to accurately meter the frozen aqueous particles 18. Carrier gas flow may be used to keep the frozen aqueous particles 18 fluidized. The appropriate parameters should be maintained to enable the desired flow of the frozen aqueous particles 18 through the delivery mechanism 100 and into the target area of the respiratory system. The volume of frozen aqueous particles 18 in the hopper reservoir 120 and the volume/flow rate/pressure/composition of fluidizing gas may be adjusted to obtain the desired flow characteristics of the frozen aqueous particles 18 into the delivery chamber 150. The hopper 110 and/or hopper reservoir 120 may be controlled to one or more pressures and maintained at, or at a higher or lower level than ambient/atmospheric. In addition to or as an alternative, mechanical agitation, such as a stirring mechanism and/or vibration of the hopper 110 and/or hopper reservoir 120 and/or other components of the delivery mechanism 100 may be used to fluidize or assist in the fluidization of the frozen aqueous particles 18.

A hopper reservoir isolation valve 130, which may enable metering, may be used to separate the hopper reservoir 120 from the delivery chamber 150. The hopper reservoir isolation valve 130 may be used to enable pressurization (continuous or intermittent, positive or negative) of any of the hopper 110, hopper reservoir 120, delivery chamber 150, as well as to meter frozen aqueous particles 18 from the hopper reservoir 120 into the delivery chamber 150. Metering may be accomplished, for example, by modulating the time and/or the percentage open of the hopper reservoir isolation valve 120 so that a specific amount of frozen aqueous particles 18 are transferred into the delivery chamber 150. Metering may be gravity fed and/or pneumatic, assisted with positive or negative pressure, and/or with mechanical assistance.

The delivery chamber 150 is used to receive a metered bolus of frozen aqueous particles 18, from the hopper reservoir 120, to be delivered to the respiratory system. The delivery chamber 150 may be such that its size and shape predominantly affect the amount of frozen aqueous particles 18 it will receive, effectively metering the bolus. The delivery chamber 150 will generally be adjacent to the hopper 110 and separated by the hopper reservoir isolation valve 130. Alternatively, the hopper isolation valve 130 may be integrated into the hopper reservoir 120 or the delivery chamber 150. The delivery chamber 150 may also serve to directly receive the frozen aqueous particles 18 from the frozen aqueous particle generator 1, or from storage, without the need for a hopper 110 and/or hopper reservoir 120. The delivery chamber 150 may be pressurized prior to and/or during delivery by a carrier gas, such as air, gases containing a different percentage of oxygen than air, gaseous drugs, gas(es) containing particulates, etc. The carrier gas may be matched in oxygen concentration to a ventilator oxygen percentage. The carrier gas may be introduced into the delivery chamber in a regulated manner (flow, pressure, continuous, intermittent, etc.) to pressurize the delivery chamber 150. The carrier gas may be introduced into the delivery chamber 150 through fluidizing diffusers 140 and be used to fluidize the frozen aqueous particles 18. The carrier gas may be used to transport the frozen aqueous particles 18 from the delivery chamber 150 to the patient. The delivery chamber may also have a reduction in pressure to assist in moving the frozen aqueous particles 18 from the hopper reservoir 120 into the delivery chamber 150.

A line fluidizer 170 may be incorporated to inject gas or other materials into the frozen aqueous particles 18 during delivery. This may be done to maintain or adjust the fluidization, particle distribution/bulk density, temperature, gas/material make up, etc. The line fluidizer 170 may be radial, linear, or other configurations suitable to achieve the desired delivery properties.

A patient isolation valve 190 may be located between the delivery chamber 150 and the patient interface 200. The patient interface valve 190 is opened to allow delivery of the frozen aqueous particles 18 from the delivery chamber 150 into the patient. The patient interface valve 190 or delivery chamber 150 may also used to prevent/reduce any potential transfer of gases, humidity, liquids, biologic materials, etc. from the patient back into the patient interface 200 or delivery chamber 150. This is desirable to preserve sanitary hardware and prevent the properties of and/or changes in delivery of the frozen aqueous particles 18 to the respiratory system. The patient isolation valve 190 or another inline valve, may be used to control pressurization and discharge of the delivery chamber 150.

The delivery pathway may include one or more tapers 180 or sections with increases or decreases in cross-sectional area or tortuosity or construction/materials that may be used to increase or decrease the bulk density of the frozen aqueous particles 18 and/or their relative position and distribution within the transfer tube/patient interface. One or more tapers 180 may be used to decrease the cross-sectional area through which the frozen aqueous particles 18 are transported. By decreasing the cross-sectional area, the frozen aqueous particles 18 may be compacted, increasing the bulk density of the delivered frozen aqueous particles 18. Such a taper(s) 180 may be used to move the transport phase from, for example, from a dilute phase to a dense phase, dense phase—dune flow to dense phase—plug flow, etc. Conversely, a taper(s) 180 may have an increase in cross-sectional area and be used to decrease the bulk density of the delivered frozen aqueous particles 18. In this case, for example, the frozen aqueous particles 18 delivered could be moved from dense phase—plug flow to dense phase—dune flow.

Depending on the geometry of the delivery chamber 150 and downstream features, it is possible to shift delivery from a dense phase—plug flow towards dense phase—dune flow or dilute phase—strand flow by modulating how the frozen aqueous particles 18 fill the delivery chamber 150 and/or the patient interface 200. If the frozen aqueous particles 18 do not fill the delivery chamber 150 completely, carrier gas may rush past/over the frozen aqueous particles 18, dragging the frozen aqueous particles 18 along with it, resulting in dense phase—dune flow or dilute phase—strand flow. It is possible to shift dense phase—dune flow or dilute phase—strand flow towards or to dense phase—plug flow by including one or more tapers 180 or features to increase the bulk density of the frozen aqueous particles 18. One way to modify transport is to increase the flow rate locally, for example, using a nozzle in the delivery chamber 150 to entrain the frozen aqueous particles 18 into the carrier gas. At the same flow rate, different openings (or a variable opening) may be made to deliver dilute phase (aerosol) through dilute phase—strand flow, dense phase—dune flow, and dense phase-plug flow delivery.

Timing of the delivery of the frozen aqueous particles 18 may be used to change the delivery of frozen aqueous particles 18 in multiple ways. Opening of the patient isolation valve 190 may have an effect on transport phase due to the backpressure seen from the lungs. Opening the patient isolation valve 190 earlier in the breathing cycle may deliver frozen aqueous particles 18 while the bronchial tree is less inflated. Opening the patient isolation valve 190 prior to or after the peak of inhalation, that is, later in the breathing cycle, has the effect of delivering frozen aqueous particles 18 while the bronchial tree is fully inflated during the peak of inhalation or even after peak inhalation. Depending on the transit time from the frozen saline particles through the remaining system components, out the patient interface, and to the target tissue, the timing of opening of the patient isolation valve 190 may be used to optimize/modify, for example, the target region(s), deposition/distribution characteristics, flow/transport phase characteristics, the quantity/type of material that may be delivered, etc.

Another way to affect transport phase is to pre-charge the delivery chamber 150 with gas and allow it to discharge into the patient. This tends to move the transport phase towards a more dilute or dense phase—dune flow rather than dense phase—plug flow that is created by pneumatically pushing the delivery of the frozen aqueous particles 18 with the carrier gas. Either or both pushing and discharging/depressurizing into the patient may be used depending on target region(s), deposition/distribution characteristics, flow/transport phase characteristics, the quantity/type of material that may be delivered, etc.

The transport phase of the frozen aqueous particles 18, or drugs/biologics, etc. may be adjusted to provide for targeted delivery to the trachea, upper airways, distal bronchial tree, alveoli—or combination thereof. For example, for delivery of frozen aqueous particles 18, particularly, frozen normal saline, to the distal bronchial tree and alveoli, it may be desirable to deliver individual boluses of frozen normal saline of 0.1 g to 10 g, and more preferably, 0.1 g to 3 g in dense phase—plug flow with typically less than 6 L, preferably less than 1 L, and more preferably 0.5 L or less of carrier gas A flow restrictor proximal (for example, a taper 180) to the delivery chamber 150 may be incorporated such that that flow is normalized before it reaches the frozen aqueous particles 18, whereas for a drug/biologic targeted to the trachea or upper bronchial tree, a transport phase more towards dilute phase may be targeted using similar flows and pressures but placing the flow restriction (for example, a taper 180) just before the delivery chamber 150 so that high local velocities entrain the frozen aqueous particles 18 to be delivered. In certain embodiments, a single or multiple larger boluses of up to 100 grams, may be transported into the respiratory system.

The diameters and sizes of the patient interface 200, taper(s) 180, delivery chamber 150, or other system components may be adjusted and optimized to affect the desired transport phase by changing, for example, dimensions, locations, etc.

In certain embodiments, multiple particle generators, system components, and/or delivery mechanisms 100 may be used for multiple source materials. Multiple source materials may be combined prior to or during delivery, delivered discretely or delivered together. Examples include multiple source materials being fed into a common hopper 110, and delivered together; multiple source materials each being fed into a delivery mechanism 100 and delivered at different or the same time point; multiple source materials and multiple delivery mechanisms 100 delivering material through one or more patient interfaces 200 to one or more target regions.

As can be seen, the system is capable of or can be configured for producing, delivering, and adjusting/modifying a multitude of parameters, including multiple source materials, to achieve the desired quantity of material delivered to the targeted region, safely and effectively.

The patient interface 200 preferably extends from the patient isolation valve 190 and may be configured as a tube, such as an endotracheal tube, or component that is integral with or connects to another component and allows extension into the patient's trachea, such as an endotracheal tube, and/or it may be connected to or part of a mask, nasal tube, or breathing apparatus. The patient interface 200 and the delivery tube may be one and the same component. The patient isolation valve 190, if used, may be located anywhere downstream of the delivery chamber 150. The patient interface 200 is used to transport the frozen aqueous particles 18 to or towards the patient. The patient interface 200 or other system component (e.g. patient isolation valve 190) or external component (e.g. an endotracheal tube if distinct from the patient interface 200) may be used in conjunction with a separate or integrated ventilator 300 to provide breathing gas, anesthesia, drugs, etc. to the patient.

The delivery of frozen aqueous particles 18 to the patient may be metered by volume, weight, density, etc. The amount, delivery of, and composition of carrier gas may be adjusted including but not limited to changes in oxygen concentration, inclusion of any drugs (anesthesia, bronchodilators) and/or particulates, pressure, volume, flow rate, velocity, etc. The fluidizing gas similarly may be similarly adjusted. Any of the gases may be adjusted or used at a selected temperature, such as cooling the fluidizing gas(es) and/or carrier gas(es). The delivery of frozen aqueous particles 18 as well as all other operating parameters may be adjusted and metered between individual deliveries (e.g. breadth to breadth), over a specific timeframe, preset, or variable.

Any or all of the operating parameters of the system may be controlled or modified by the system controller 195 e.g. under software control, for example, via one or more algorithms which may or may not receive input(s) from other equipment, such as a ventilator, patient monitor/monitoring equipment, database, computer(s), etc., with or without physician input, or some combination thereof. The controller 195 may be analog. The controller 195 may be connected to and operate and/or interface with, for example, valves, delivery mechanism 100, frozen aqueous solution particle generator 1, material flow/movement, temperature/humidity management, pressures, grinding, milling, fluidization, stirring, ventilation/ventilator, etc. The controller 195 may use a suitable user interface 197, such as a touchscreen, manual/analog controls, a combination thereof, etc. Delivery of frozen aqueous particles 18, drugs/biologics/etc. may be every breadth or may skip breadths. These parameters may be modified based on changes in patient physiologic parameters, system operating requirements (for example, temperatures, power, etc), local conditions (for example, relative humidity), desired therapy for the patient, etc. The delivery phase and/or bulk density may be different throughout the system, e.g. the material may leave the metering chamber 152 in one phase, e.g. dilute or dense phase—dune flow, and then be changed to another phase elsewhere in the system, e.g. increased bulk density due to a taper(s) 180 or other feature(s), resulting in dense phase—plug flow into the patient interface 200 and into the patient. Delivery phase change(s) and/or changes in bulk density may be a shift towards a more dense phase, a shift towards a more dilute phase, and/or an increase or decrease in bulk density. For example, the frozen aqueous particles 18 may leave the delivery chamber 152 in dense phase—plug flow at one bulk density and then pass through a taper 180 which increases the bulk density while maintaining dense phase—plug flow. The patient interface 200 and/or other system component may have a taper(s) 180 or other feature(s) that increases or decreases bulk density locally, resulting in a different delivery phase. The transition between patient interface 200 and patient anatomy may also be used to optimize the phase or distribution of frozen aqueous particles 18 across the cross section of the patient interface 200 or anatomy.

Any or all of the components of the system, such as but not limited to the hopper 110, hopper reservoir 120, hopper reservoir isolation valve 130, carrier gas delivery valve 160, patient isolation valve 190, delivery chamber 150, metering chamber 152, taper(s) 180, and patient interface 200 may be environmentally controlled and/or sealed. It may be desirable to maintain a specific temperature and/or level of humidity of/within the hopper 110, hopper reservoir 120, hopper reservoir isolation valve 130, carrier gas delivery valve 160, patient isolation valve 190, delivery chamber 150, metering chamber 152, taper(s) 180, and patient interface 200 at a temperature and/or humidity near or at that of the frozen aqueous particles 18 to reduce the likelihood of frozen aqueous particle 18 agglomeration and/or negative effects on delivery. Temperature and/or humidity levels may vary from component to component, as well as for fluidizing and carrier gases, etc. For example, for delivery of frozen saline, humidity is controlled primarily with temperature in the chambers and the patient interface 200. Carrier and/or fluidizing gases may be maintained at a low enough humidity that frozen aqueous particle 18 buildup or degradation over the intended operating time is negligible.

An additional way that humidity and/or temperature may be controlled is by preventing the back flow of warm humid air from the patient into the patient interface 200 or other system components, such as by using a patient isolation valve 190. Fluidization gas may also be used as a purge to maintain low temperature and/or humidity levels in the chamber(s) and/or material if ambient moisture migration is a hazard to either the material (particle) properties or the chemical stability of the material.

Any components of the system may be integrated with each other as well as be separate components. For example, the hopper 110 and hopper reservoir 120 may be a single component. The frozen aqueous particle generator 1 and the delivery mechanism 100 may be a single component. The system may be integrated with, encompass, connect with, or use a separate ventilator or other equipment.

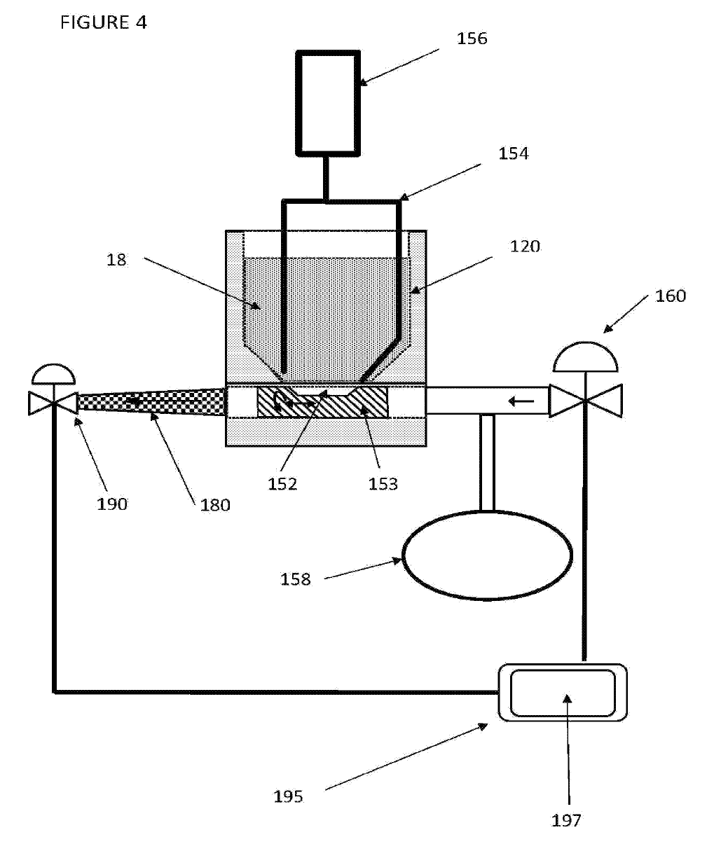

An alternate embodiment to the delivery mechanism 100 is shown in FIG. 4. In FIG. 4, the frozen aqueous particles 18 in the hopper reservoir 120 are fluidized by agitation from a stirring mechanism 154, which is driven by a motor 156. These fluidized frozen aqueous particles 18 are then directly fed into a metering chamber 152 with the desired bolus for delivery. The metering chamber 152 is formed in conjunction with or by the bolt 153. The bolt 153 may be rotary, linear, or a combination thereof to accept the frozen aqueous particles 18. Actuation of the bolt 153 may be pneumatic, motor driven, or any type or combination of operation to enable introduction of frozen aqueous particles 18 into the metering chamber 152. The amount the bolt 153 opens, the time over which it is opened, the fill height of the hopper 110, the differential pressure between the hopper 110 and the delivery chamber 150, duration, timing or magnitude of fluidizing parameters such as gas flow or vibration, or some combination of these or other parameters may be used to flow and meter the quantity of frozen aqueous particles 18 loaded into the delivery chamber 150. Additionally, the bolt 153 may be used to isolate the hopper reservoir 120. In this example, the carrier gas delivery valve 160 and patient isolation valve 190 open to allow frozen aqueous particles 18 to be transported down the patient interface 200 and into the patient's respiratory system. The hopper reservoir 120 and/or metering chamber 152 may be pressurized (positive and/or negative) continuously or intermittently or not at all. A pneumatic cylinder 158 or other suitable components may be incorporated to draw back or pull negative pressure during delivery of frozen aqueous particles 18 into the metering chamber 152. The decreased pressure assists in moving the frozen aqueous particles 18 from the hopper reservoir 120, may be used to fluidize it locally, and enable more frozen aqueous particles 18 to flow into the metering chamber 152. Additionally, a taper 180 and patient isolation valve 190 may be incorporated.

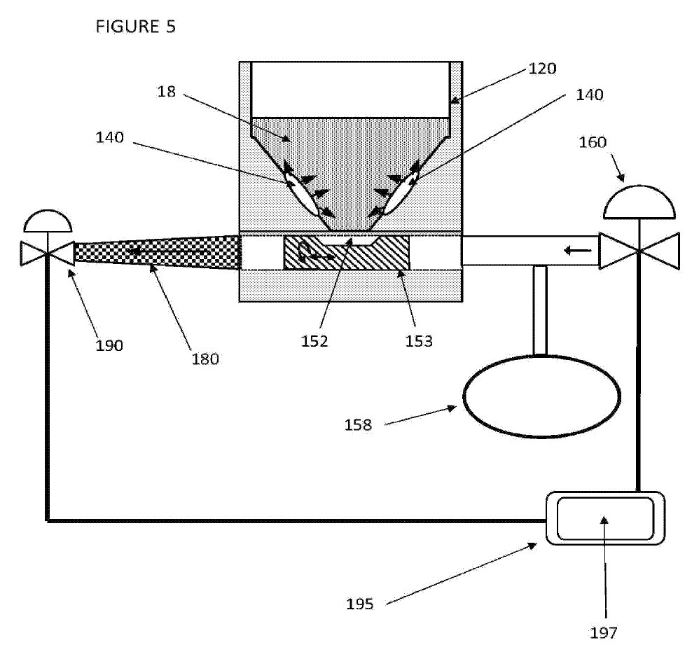

An alternate embodiment to the delivery mechanism 100 is shown in FIG. 5. In FIG. 5, the frozen aqueous particles 18 in the hopper reservoir 120 are fluidized through fluidization diffusers 140. These fluidized frozen aqueous particles 18 are then directly fed into a metering chamber 152 with the desired bolus for delivery. The metering chamber 152 is formed in conjunction with or by the bolt 153. The bolt 153 may be rotary, linear, or a combination thereof to accept the frozen aqueous particles 18. Actuation of the bolt 153 may be pneumatic, motor driven, or any type or combination of operation to enable introduction of frozen aqueous particles 18 into the metering chamber 152. The amount the bolt 153 opens, the time over which it is opened, the fill height of the hopper 110, the differential pressure between the hopper 110 and the delivery chamber 150, or some combination of these or other parameters may be used to meter the quantity of frozen aqueous particles 18 loaded into the delivery chamber 150.

Additionally, the bolt 153 may be used to isolate the hopper reservoir 120. In this example, the carrier gas delivery valve 160 and patient isolation valve 190 open to allow frozen aqueous particles 18 to be transported down the patient interface 200 and into the patient's respiratory system. The hopper reservoir 120 and/or metering chamber 152 may be pressurized (positive and/or negative) continuously or intermittently or not at all. A pneumatic cylinder 158 or other suitable components may be incorporated to draw back or pull negative pressure during delivery of frozen aqueous particles 18 into the metering chamber 152. The decreased pressure assists in moving the frozen aqueous particles 18 from the hopper reservoir 120, may be used to fluidize it locally, and enable more frozen aqueous particles 18 to flow into the metering chamber 152. Additionally, a taper 180 and patient isolation valve 190 may be incorporated.

A representative example of a method for delivering material to the respiratory system in a higher density phase compared to dilute phase transport (aerosol) can include one or more of the following steps. In this example, the aqueous solution will be normal (0.9%) saline. Liquid saline is obtained in 1 L bags in sufficient quantity for the procedure. The liquid saline is introduced into the reservoir 20. The liquid saline flows down past the thermoelectric/electromechanical cooler 30 and is reduced in temperature sufficient to phase change the liquid saline into frozen saline. The frozen saline is fed into a course grinder 40, and ground into the course ground frozen saline in pieces nominally less than 10 mm in size, preferably less than 5 mm. The grinding takes place at a sufficiently low temperature to enable freezing and prevent accumulation of atmospheric moisture. For example, at −21.1° C. or lower, which is the minimum freezing temperature for NaCl in an aqueous solution. The course ground frozen saline may be sorted for particle size or other physical parameter(s). The course ground frozen saline is transferred to a mill 50 and passed through a mill cutter 52, where the course ground frozen saline is reduced in size to nominally less than 2000 μm, more preferably less than 500 μm, with an aspect ratio of nominally not more than 5:1, preferably 3:1. The hopper 110 and/or hopper reservoir 120 may be sufficiently environmentally controlled to prevent degradation of the frozen saline particles, such as below 0° C., with minimal humidity. The frozen saline particles in the hopper 110 and/or hopper reservoir 120 are optionally fluidized constantly or intermittently with gas, such as air, which may be cooled. The frozen saline particles pass the hopper reservoir isolation valve 130 and are metered into the delivery chamber 150. The delivery chamber 150 is sufficiently environmentally controlled to prevent degradation of the frozen saline particles, such as below 0° C., with minimal humidity. The frozen saline particles in the delivery chamber 150 are optionally fluidized constantly or intermittently with gas, such as air, which may be cooled.

The carrier gas delivery valve 160 is opened and the delivery chamber 150 is charged with the carrier gas, such as air or a gas similar to that which is delivered through the ventilator. The frozen saline particles pass through a taper 180 which increases the bulk density of the frozen saline particles. The patient isolation valve opens and the frozen saline particles flow into the patient interface 200. The frozen saline particles pass through the patient interface 200 in dense phase—plug flow and then into the patient's lungs, where the frozen saline particles are preferably deposited in the distal bronchial tree and alveoli.

The foregoing description of various embodiments of the invention has been presented for purposes of illustration and description. It is not intended to limit the invention to the precise forms disclosed. Many modifications, variations and refinements will be apparent to practitioners skilled in the art. For example, embodiments of the device may be sized and otherwise adapted for various pediatric applications as well as various veterinary applications. Also, those skilled in the art will recognize, or be able to ascertain using no more than routine experimentation, numerous equivalents to the specific devices and methods described herein. Such equivalents are considered to be within the scope of the present invention and are covered by the appended claims below. Elements, characteristics, or acts from one embodiment may be readily recombined or substituted with one or more elements, characteristics or acts from other embodiments to form numerous additional embodiments within the scope of the invention. Moreover, elements that are shown or described as being combined with other elements, can, in various embodiments, exist as standalone elements. Hence, the scope of the present invention is not limited to the specifics of the described embodiments, but is instead limited solely by the appended claims.

Unlike in aerosols, particles in dense phase transport are not suspended by gas. Particle interactions are responsible for much of the bolus support in dense phase transport. Because of this, conventional restrictions on particle size, mass, density, and shape may be reduced or eliminated.

Although room temperature powders could potentially be delivered by an uncooled system similar to the one described later, it is currently believed that physiologic parameters would prevent the successful delivery of dry powders in such large quantities. Dry powders delivered in dense phase may overwhelm the lungs by rapidly reducing moisture content and potentially dehydrating the pulmonary system. Therefore, a cryogenic (multi-phase) particle generation system will be necessary. This particle generation system can be as described in previous Qool Therapeutics patents or IP disclosures; alternatively, it may make use of ball mill, jet mill, or any other cryogenic comminution (cryogenic micronization) technology available. If it is necessary to deliver large amounts of dry material via dense phase, this could be accomplished by supplying a mixture of the drug and frozen saline preventing dehydration of the pulmonary system.

Though the mechanics governing dense phase transport will enable the theoretical use of a wider range of particle sizes, densities, and other physical properties, tuning a powder or powder mixture will be critical to targeted delivery. Mixtures of particles with different particles size distributions, cohesive properties, morphology (spherical shape versus snow flake shape) will all likely change the modes of deposition in the lungs, bronchial tree, and upper airways enabling multiple paths for targeted treatments.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

All references, including publications, patent applications, and patents, cited herein are hereby incorporated by reference to the same extent as if each reference were individually and specifically indicated to be incorporated by reference and were set forth in its entirety herein.

1. A method for producing a series of frozen solid particle (FSP) boluses for delivery to a patient interface, said method comprising:
(a) providing a source of FSP;
(b) transferring a metered bolus of FSP from the source into a delivery chamber;
(c) fluidizing the metered bolus of FSP;
(d) transferring the fluidized metered bolus of FSP to the patient interface; and
(e) repeating steps (b) through (d) to deliver a series of discrete FSP boluses into the patient interface.

2. A method as in claim 1, wherein providing the source of FSP comprises storing a volume of preformed FSP in a hopper.

3. A method as in claim 1, wherein providing the source of FSP comprises comminuting an aggregate source of frozen material in FSP.

4. A method as in claim 1, wherein providing the source of FSP comprises freezing a liquid source material into FSP in situ.

5. A method as in claim 1, wherein transferring the metered bolus of FSP into a delivery chamber comprises flowing the FSP from the source into the delivery chamber and metering with a valve.

6. A method as in claim 5, wherein the valve is integrated with the delivery chamber.

7. A method as in claim 5, wherein flowing the FSP from the source into the delivery chamber comprises at least one of gravity flow and differential pressurization of the source and delivery chamber.

8. A method as in claim 7, wherein flowing the FSP from the source into the delivery chamber comprises both gravity flow and differential pressurization of the source.

9. A method as in claim 1, further comprising transferring the metered bolus of FSP from the delivery chamber to the patient interface through a transfer tube.

10. A method as in claim 9, wherein fluidizing the metered bolus of FSP comprises fluidizing within at least one of the delivery chamber, the transfer tube, and the patient interface.

11. A method as in claim 9, wherein fluidizing the metered bolus of FSP comprises fluidizing within each of the delivery chamber, the transfer tube, and the patient interface.

12. A method as in claim 9, wherein the delivery chamber and the transfer tube are integrated into a single unit.

13. A method as in claim 1, further comprising controlling the conditions of fluidizing the metered bolus of FSP and of transferring the fluidized metered bolus of FSP to the patient interface in order to control a density of the FSP delivered to the patient interface.

14. A method as in claim 13, wherein the conditions are controlled by a controller.

15. A method as in claim 14, wherein the conditions are controlled to cause a strand flow to the patient interface.

16. A method as in claim 14, wherein the conditions are controlled to cause a dune flow to the patient interface.

17. A method as in claim 14, wherein the conditions are controlled to cause a plug flow to the patient interface.

18. A method as in claim 9, wherein the transfer tube is tapered in the direction from the delivery chamber toward the patient interface in order to densify the FSP as they flow to the patient interface.

19-44. (canceled)

45. A system for producing a fluidized metered bolus of frozen solid particle (FSP), said system comprising:
a source of FSP;
a delivery chamber configured to receive a metered bolus of FSP from the source;
a transfer tube having an inlet end configured to receive the metered bolus of FSP from the delivery chamber and an outlet end; and
a fluidizer operatively coupled to the delivery chamber and/or the transfer tube and to transfer the fluidized metered bolus of FSP through the transfer tube.
wherein the outlet end of the transfer tube is configured to be coupled to a patient interface.

46-63. (canceled)
